(19) United States
(12) Patent Application Publication
(10) Pub. No.: US 2026/0137271 A1
(43) Pub. Date: May 21, 2026

(54) SLIT LAMP ASSEMBLY WITH IMPROVED STRUCTURE

(71) Applicant: Michael Sayonh, Tupelo, MS (US)

(72) Inventor: Michael Sayonh, Tupelo, MS (US)

(21) Appl. No.: 18/949,655

(22) Filed: Nov. 15, 2024

(51) Int. Cl. A61B 3/00; A61B 3/135

(52) U.S. Cl. CPC A61B 3/0083; A61B 3/135

(57) ABSTRACT

A slit lamp assembly with an improved structure, the slit lamp assembly including a slit lamp to allow a medical professional to observe abnormalities of an eye of a patient, and a base, including a slit lamp receiving portion disposed on a top surface of the base to receive the slit lamp thereupon, and a concave indentation cut into a rear edge portion of the base nearest the patient and having a concave shape that prevents a stomach or chest of the patient from contacting the rear edge portion of the base.

### BACKGROUND

### 1. Field

The present general inventive concept relates generally to a slit lamp and/or a slit lamp biomicroscope, and particularly, to a slit lamp assembly with an improved structure.

### 2. Description of the Related Art

In the field of optometry, a specialized profession that involves examining the eyes for defects or abnormalities, a common tool used during examination is a slit lamp (a.k.a., a slit lamp biomicroscope).

The conventional standard design of a slit lamp, however, does not optimally suit varied body types such as well-endowed females and statures resembling obesity.

Evaluating these demographics with traditional equipment makes it difficult to properly examine patients from a provider's perspective and patients may perceive they are not receiving the level the care they require.

One of the main goals of a clinician is to provide a pleasant experience for the patient while focusing on improving their vision.

However, when the patients are unable to feasibly fit into the standard equipment, this may increase the chance of patients reporting negative encounters with their provider.

Therefore, there is a need for an improved device that allows for comfortable examination of an eye for defects.

### SUMMARY

The present general inventive concept provides a slit lamp assembly with an improved structure.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a slit lamp assembly with an improved structure the slit lamp assembly including a slit lamp to allow a medical professional to observe abnormalities of an eye of a patient, and a base, including a slit lamp receiving portion disposed on a top surface of the base to receive the slit lamp thereupon, and a concave indentation cut into a rear edge portion of the base nearest the patient and having a concave shape that prevents a stomach or chest of the patient from contacting the rear edge portion of the base.

The slit lamp assembly may further include a head receiving portion, including a head rest mount disposed on the head receiving portion, a forehead rest disposed on the head rest mount to receive a forehead of the patient thereupon, and a chin rest disposed below the forehead rest to receive a chin of the patient thereupon.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates an isometric perspective view of a slit lamp assembly with an improved structure, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Slit Lamp Assembly With An Improved Structure 100
Head Receiving Portion 110
Head Rest Mount 111
Forehead Rest 112
Chin Rest 113
Base 120
Slit Lamp Receiving Portion 121
Concave Indentation 122
Forearm Receiving Portion 123
Slit Lamp 130
Eyepieces 131
Illumination Unit 132
Fixation Target 133

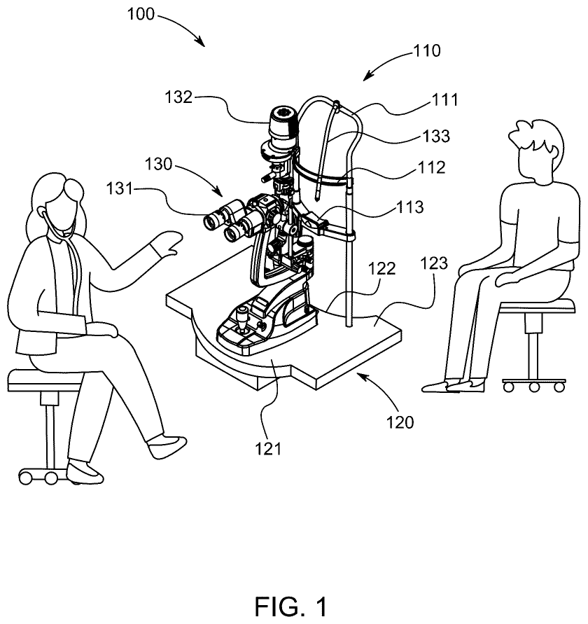

FIG. 1 illustrates an isometric perspective view of a slit lamp assembly with an improved structure 100, according to an exemplary embodiment of the present general inventive concept.

The slit lamp assembly with an improved structure 100, and all components therein and/or connected thereto, may be constructed from at least one of metal, plastic, stone, ceramics, wood, silicone, glass, paper, cloth, circuitry, leather, memory foam, latex, and rubber, etc., but is not limited thereto, and can be constructed from any material known to one of ordinary skill in the art.

Referring to FIG. 1, the slit lamp assembly with an improved structure 100 may include a head receiving portion 110, a base 120, and a slit lamp 130, but is not limited thereto.

The head receiving portion 110 may include a head rest mount 111, a forehead rest 112, and a chin rest 113, but is not limited thereto.

The head rest mount 111 may be disposed at a top and middle portion of the head receiving portion 110, and may include the forehead rest 112 disposed thereupon to receive a forehead of a patient thereupon. In other words, the patient may press his/her forehead against the forehead rest 112.

The chin rest 113 may be disposed at a middle and lower portion of the head rest mount 111, and may receive a chin of the patient thereupon while the forehead of the patient is pressed against the forehead rest 112.

The forehead rest 112 and the chin rest 113 may both be designed to be concave, such that the forehead rest 112 and the chin rest 113 may be ergonomic to allow the patient's forehead and chin to be supported with comfort to the patient.

The base 120 may include a slit lamp receiving portion 121, a concave indentation 122, and a forearm receiving portion 123, bit is not limited thereto.

The slit lamp receiving portion 121 may be a top surface of the base 120 that may receive the slit lamp 130 thereupon, and/or the slit lamp 130 may be fixed thereto.

Referring to FIG. 1, the concave indentation 122 is an indentation that is cut into the base 120, and is disposed at a rear edge portion of the base 120. In other words, the concave indentation 122 of the base 120 may be disposed at an edge of the base 120 that is located at a position of the base 120 where the patient sits.

Therefore, if the patient has a larger stomach and/or chest that an average person, the concave indentation 122 allows the patient to be comfortable, because the stomach and/or chest of the patient will not be pressed up against a back edge of the base 120, but will be disposed comfortably within the concave indentation 122.

The forearm receiving portion 123 includes portions of the top surface of the base 123 that allows the patient to place his/her forearms thereupon, for comfort.

The slit lamp 130 (a.k.a., the slit lamp biomicroscope 130) may be a conventional slit lamp 130 including eyepieces 131, an illumination unit 132, and a fixation target 133, but is not limited thereto, and may include any and all other parts, portions, and components of a conventional slit lamp known to one of ordinary skill in the art.

The present general inventive concept may include a slit lamp assembly with an improved structure 100, the slit lamp assembly 100 including a slit lamp 130 to allow a medical professional to observe abnormalities of an eye of a patient, and a base 120, including a slit lamp receiving portion 121 disposed on a top surface of the base 120 to receive the slit lamp 130 thereupon, and a concave indentation 122 cut into a rear edge portion of the base 120 nearest the patient and having a concave shape that prevents a stomach or chest of the patient from contacting the rear edge portion of the base 120.

The slit lamp assembly 100 may further include a head receiving portion 110, including a head rest mount 111 disposed on the head receiving portion 110, aforehead rest 112 disposed on the head rest mount 111 to receive a forehead of the patient thereupon, and a chin rest 113 disposed below the forehead rest 112 to receive a chin of the patient thereupon.

A main purpose of the present general inventive concept is to provide users with an improved eye examination device that meets the facial and body contour of diverse populations. Ingenious and practical, the present general inventive concept offers a modern advancement in optometric equipment designed to elevate the patient's experience while ensuring examination accuracy. Developed with a keen focus on ergonomic comfort, the present general inventive concept introduces an alternative design where the base platform allows the necessary space needed for patients of all body types to comfortably place their head into a slit lamp in the correct position for the examiner. To further enhance patient comfort, forearm rests are strategically positioned on both lateral sides of the base platform, to provide additional support as needed. The primary headrest of the present general inventive concept remains adjustable allowing for seamless adaptation to different head sizes and shapes. Retaining all standard functionalities expected from a slit lamp, including adjustable light intensity, magnification levels, and precise focusing capabilities, this improved option seamlessly integrates the power box, wiring, and mounting underneath the base platform, ensuring ease of use and accessibility for clinicians without compromising the device's ergonomic design. This innovative, top-quality product offers a design that is tailored to improve a clinician's ability to accurately examine the ocular structures for the body types mentioned above compared to the conventional slit lamp. As a result, the present general inventive concept may prove to be essential in the medical industry.

The present general inventive concept is the only product of its kind that improves optometric equipment by offering an unparalleled ergonomic slit lamp option tailored to the needs of diverse patient populations, specifically for obese body structures or well-endowed women. This unprecedented product is uniquely crafted with durability, permits ease of cleaning post-use, and was defined by an optometrist who understands comfort is luxury and is committed to providing the best patient care when leading eye examinations.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A slit lamp assembly with an improved structure, the slit lamp assembly comprising:
a slit lamp to allow a medical professional to observe abnormalities of an eye of a patient; and
a base, comprising:
a slit lamp receiving portion disposed on a top surface of the base to receive the slit lamp thereupon, and
a concave indentation cut into a rear edge portion of the base nearest the patient and having a concave shape that prevents a stomach or chest of the patient from contacting the rear edge portion of the base.

2. The slit lamp assembly of claim 1, further
comprising: a head receiving portion, comprising:
a head rest mount disposed on the head receiving portion,
a forehead rest disposed on the head rest mount to receive a forehead of the patient thereupon, and
a chin rest disposed below the forehead rest to receive a chin of the patient thereupon.
